(19) United States
(12) Patent Application Publication
(10) Pub. No.: US 2005/0119709 A1
(43) Pub. Date: Jun. 2, 2005

(54) Increased dielectric strength environment for an implantable medical device

(76) Inventors: Karl Gauglitz, Snohomish, WA (US); Blair Erbstoeszer, Kirkland, WA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/726,270

(22) Filed: Dec. 2, 2003

(51) Int. Cl. A61N 1/375

(52) U.S. Cl. 607/36

(57) ABSTRACT

A gaseous mixture including sulfur hexafluoride provides improved insulation properties for an implantable pulse generator. The mixture, including a leak tracing gas such as helium, is hermetically sealed in a biocompatible housing. In one embodiment, the mixture is maintained at a pressure greater than one atmosphere.

### TECHNICAL FIELD

This subject matter relates to implantable medical devices, and more particularly, to a gaseous mixture environment within a sealed housing of an implantable medical device.

### BACKGROUND

One particular class of implantable medical devices typically includes a bio-compatible housing and an electronic circuit configured to perform various functions. For example, an implantable defibrillator delivers an electrical pulse to a heart to aid in maintaining the normal pumping action. The energy level of the electrical pulse is developed, in part, by a storage capacitor and battery within the medical device.

For medical purposes, it is desirable to minimize the physical size, or volume, of the implantable medical device. The volume of an implantable medical device is often a function of the capacitor size, battery and an electronic circuit.

What is needed is a device having a reduced volume that delivers the desired electrical performance.

### SUMMARY

In one embodiment, an electronic circuit is enclosed in an hermetically sealed housing filled with a gas mixture of sulfur hexafluoride. The housing includes a welded metal container. The electronic circuit includes, for example, a defibrillator having a capacitor, battery and other electrical components. Other gases are included in the mixture, including, for example, helium, nitrogen, oxygen and argon.

Within the housing, a relationship exists between device spacing, the breakdown voltage and the composition of the gaseous environment. The device spacing and breakdown voltage are determined as a function of the gas selected for the environment.

A gas is introduced into the housing using a filler tube or port affixed to a portion of the housing. A vacuum applied to the port is used to evacuate the interior prior to introduction of a gas mixture.

Other aspects of the invention will be apparent on reading the following detailed description of the invention and viewing the drawings that form a part thereof.

### BRIEF DESCRIPTION OF THE DRAWINGS

In the drawings, like numerals describe substantially similar components throughout the several views. Like numerals having different letter suffixes represent different instances of substantially similar components.

FIGS. 1A and 1B include views of an implantable medical device according to one embodiment.

FIG. 2 includes a sectional view of a portion of a circuit.

FIG. 3 includes a partial sectional view of an electrical component within a conductive housing.

FIG. 4 includes a system for fabricating an embodiment of the present subject matter.

FIG. 5 includes a flow chart of a method according to one embodiment.

### DETAILED DESCRIPTION

In the following detailed description, reference is made to the accompanying drawings that form a part hereof, and in which is shown, by way of illustration, specific embodiments in which the present subject matter may be practiced. These embodiments are described in sufficient detail to enable those skilled in the art to practice the subject matter, and it is to be understood that the embodiments may be combined, or that other embodiments may be utilized and that structural, mechanical, logical and electrical changes may be made without departing from the scope of the present subject matter. The following detailed description is, therefore, not to be taken in a limiting sense, and the scope of the present subject matter is defined by the appended claims and their equivalents.

For a particular operating voltage and voltage margin (or guard voltage), the minimum spacing between different electrical potentials, and thus, the size or volume of an implantable medical device, is determined as a function of the insulating properties of the environment within the device. For example, one particular medical device having an atmosphere of primarily nitrogen provides a minimum breakdown voltage (operating voltage) of 2300 volts using a 1300 volt margin (design breakdown voltage is 1000 volts) when device spacing is maintained at 0.01 inches. When the same device is backfilled using primarily sulfur hexafluoride (rather than nitrogen) the minimum breakdown voltage rises to 3100 volts and thus, the design breakdown voltage increases to 1800 volts. Alternatively, if the minimum breakdown voltage is maintained at 2300 volts and the guard voltage is maintained at 1300 volts (design breakdown voltage is 1000 volts), then the device spacing can be reduced to 0.004 inches when backfilled with sulfur hexafluoride and thus the device can be fabricated in a smaller housing. Different combinations of voltages and device spacing are also contemplated.

In one embodiment, the gas mixture includes residual gases in the amount of approximately 5% oxygen, 5% helium and 20% argon. The balance of the gas mixture includes an insulating gas, examples of which include nitrogen, sulfur hexafluoride and others described elsewhere in this document.

In one embodiment, the electronic circuit includes reduced spacing between different electrical potentials. For example, different electrical potentials may be present on signal traces disposed on a circuit board, a capacitor, a portion of a housing, a battery or other structure within an implantable medical device.

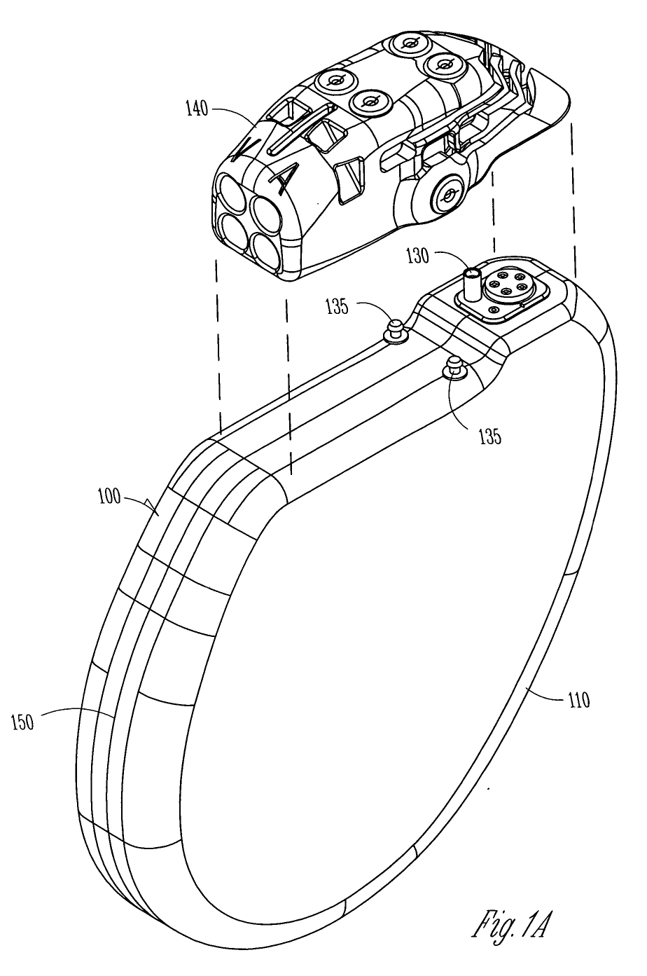

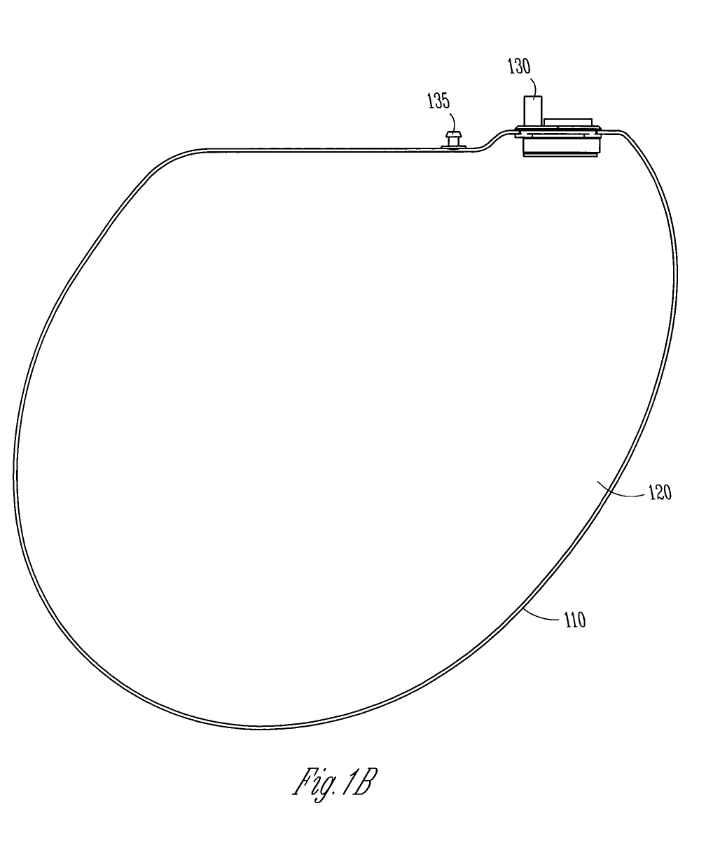

FIGS. 1A and 1B illustrate views of housing 100 according to one embodiment. In the figures, housing 100 includes an hermetically sealed container having wall 110 enclosing interior 120. Backfill locations include port 130 and anchor post 135, each of which are affixed to a portion of wall 110 and includes a structure for directing a gas into, and from, interior 120. Port 130 includes a tubular structure that carries a mounting plate having feedthrough ports for electrical conductors. According to one embodiment, to seal a gas within interior 120, a metal plug, or ball, is inserted and welded in a lumen of port 130. According to one embodiment, to seal a gas within interior 120, anchor post 135 is inserted and welded into an orifice of wall 110. Two anchor posts 135 are illustrated in the figure, however, more or less than two are also contemplated. In addition, other structures are contemplated for introducing a gas into, and evacuating a gas from, housing 100.

FIG. 1A illustrates partially assembled housing 100 having two wall sections 110 joined by welded joint 150. In one embodiment, joint 150 is laser welded in an atmosphere of inert gas. Header 140 is adhesively mounted on housing 100 and is aligned by anchor pins 135. Header 140 provides electrical connections to an electronic circuit within housing 100. One or more electrical leads emanate from header 140 of housing 100. In one embodiment, at least a portion of housing 100 is electrically conductive and, when implanted in a body, provides an electrical connection with a particular organ or tissue.

FIG. 1B illustrates a view of a wall section 110 according to one embodiment. In the figure, anchor pin 135 and tube 130 are affixed to wall section 110. Wall section 110, in one embodiment, is fabricated of stamped sheet metal. In one embodiment, housing 100 is fabricated of titanium or a titanium alloy. An electronic circuit is disposed within interior 120.

FIG. 2 illustrates a sectional view of a portion of an electrical circuit disposed within interior 120 according to one embodiment. In the figure, circuit board 200 includes conductive circuit trace 210 and trace 220 separated by distance A. Circuit board 200 is fabricated of insulative material and, in various embodiments, includes a flexible or rigid material and is sometimes referred to as a substrate.

Traces 210 and 220 connect selected electrical components within housing 100. In various embodiments, the electrical components are disposed on circuit board 200 and other structures of housing 100. Representative electrical components include batteries, capacitors, microprocessors, resistors, connectors and other such components. Traces 210 and 220 carry electric current.

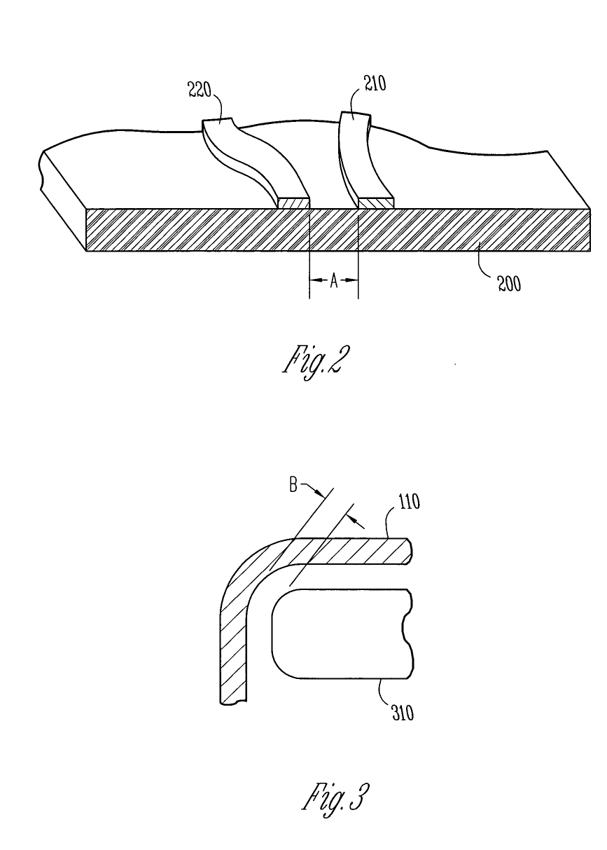

FIG. 3 illustrates a portion of wall 110 near component 310. Component 310 is disposed within an interior of housing 100 and is at a first electrical potential and wall 110 is at a second electrical potential. Distance B provides separation between wall 110 and component 310. Component 310, in various embodiments, includes a capacitor, a battery, or other electrical component.

Distance A and distance B are selected to provide physical separation between different electrical potentials present within housing 100. In various embodiments, distance A and distance B are each approximately 0.004 to 0.01 inches. Dimensions for each of distance A and distance B of less than 0.004 inches and greater than 0.01 inches are also contemplated. The spacing between electrically conductive paths, examples of which include that denoted as distance A and distance B, is sometimes referred to as device spacing.

The dielectric properties of the environment between the current carrying conductors separated by a gap or space is a function of the gas within that gap or space. For example, with an insulating gas of sulfur hexafluoride, a space of at least 0.004 inches between conductors allows a minimum breakdown voltage of 2300 volts and a margin of 1300 volts.

The net effect of increasing the dielectric strength of the environment within an implantable medical device includes at least one of any combination of (a) increased operating voltage; (b) increased voltage margin; and (c) reduced device spacing. The dielectric strength can be increased by elevating the pressure in the implanted medical device and by selecting an insulating gas having a dielectric constant greater than that of nitrogen. Exemplary gases and their dielectric constants are presented elsewhere in this document.

For example, one particular medical device having an atmosphere of primarily nitrogen at a pressure of approximately 0.1 pounds per square inch provides a minimum breakdown voltage (operating voltage) of 3100 volts when device spacing is maintained at 0.01 inches. When the pressure is increased to approximately 5 pounds per square inch, the same device exhibits a breakdown voltage of approximately 3700 volts.

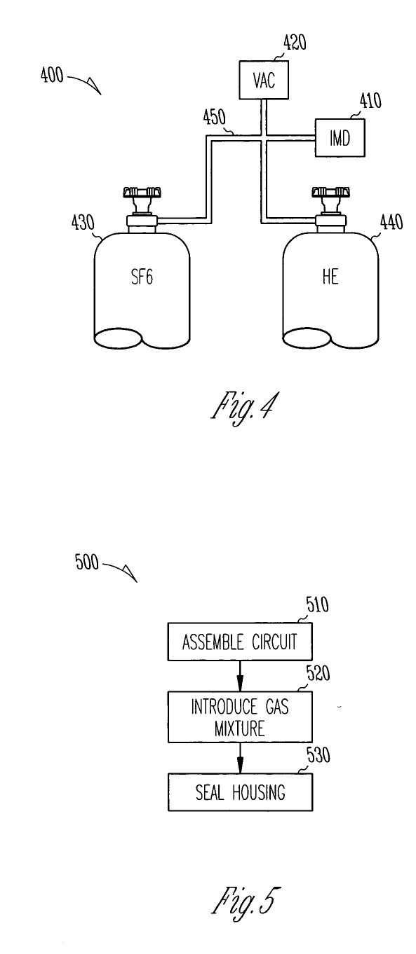

FIG. 4 illustrates system 400 for fabricating implantable medical device 410. Manifold 450 is coupled to device 410 as well as vacuum source 420, and tank 430 and tank 440. Manifold 450, in various embodiments, includes tubular conduits, valves, gauges, dryers, pumps and actuators some of which are illustrated in the figure. Device 410, in one embodiment, includes housing 100 and is coupled to manifold 450 using a backfill port such as shown at tube 130 or anchor pin 135. Vacuum source 420 includes a pump configured to draw pressure below that of 1 atmosphere (atm) and thereby exhaust the interior of device 410. Tank 430 includes sulfur hexafluoride. Tank 440 includes helium. Other tanks, manifold structures and gases (including at least one of any combination of helium, nitrogen, oxygen and argon) are also contemplated.

FIG. 5 illustrates method 500 according to one embodiment. At 510, the electronic circuit is assembled. Assembly, in various embodiments, includes soldering, mounting and attaching various electrical components as well as testing of selected components of the circuit. At 520, a gas mixture is introduced into housing 100. In one embodiment, the gas mixture includes a mixture of sulfur hexafluoride. At 530, the housing is sealed to maintain the environment within the housing. Sealing the housing, in one embodiment, includes forming a welded joint around a perimeter of the housing as well as closing off a backfill location. In one embodiment, the welded joint is formed by laser welding in an atmosphere of inert gas such as, for example but not limited to, argon. In one embodiment, closing off a backfill location includes placing a metal ball within an orifice and welding the ball in position. The ball is welded in an atmosphere of inert gas. In one embodiment, the gas mixture within the housing is maintained at a positive pressure greater than 1 atm. In one embodiment, the gas mixture is maintained at a positive, non-zero pressure greater than zero pounds per square inch gage. In one embodiment, gas pressure is maintained at a pressure of approximately 5.0 pounds per square inch gage. In one embodiment, gas pressure is maintained at a pressure greater than approximately 5.0 pounds per square inch gage.

In one embodiment, an insulating gas such as, for example but not limited to, sulfur hexafluoride is introduced into the housing after a sequence of purging or flushing operations. For example, in one embodiment, following assembly of the electronic circuitry and other components in the housing, a nitrogen gas mixture is injected and the housing is welded in an atmosphere of argon. A vacuum is then applied to draw off the nitrogen environment in the housing. The vacuum, in one embodiment, is applied by a rubber hose connected to a backfill port. A mixture of sulfur hexafluoride and helium is then injected into the housing followed by sealing of the backfill port.

The present subject matter also includes testing of the electronic circuitry in an atmosphere of a mixture of sulfur hexafluoride prior to welding of the housing. For example, in one embodiment, the electronic circuitry is assembled on a flexible substrate and prior to folding the circuitry for placement in the housing, the circuitry is tested in a fixture. The fixture includes an atmospheric chamber having a mixture of sulfur hexafluoride.

In one embodiment, the gaseous environment within the device includes between approximately 1 and 70 percent sulfur hexafluoride by volume. Variations in the amount of sulfur hexafluoride are also contemplated including amounts less than 1 percent as well as those greater than 70 percent. Approximate percentages are presented here to allow for variations in measurement and instrument accuracy as well as allow for temperature variations.

It will be appreciated that throughout this document, sulfur hexafluoride is referenced herein as an exemplary insulating gas. Sulfur hexafluoride is a non-metal halide and is relatively inert, non-toxic, non-flammable and has good cooling properties. In addition to the insulating gas, other gases are included in the mixture, including, for example, helium, nitrogen, oxygen and argon.

Insulating gases other than sulfur hexafluoride are also contemplated, including those gases enumerated in Table 1, the data of which is drawn from CRC Handbook of Chemistry and Physics, ©2000 by CRC Press LLC and incorporated herein by reference. In addition to a high dielectric strength, suitable insulating gases also exhibit bio-compatibility, inertness, non-toxicity and inflammability. In the table, the approximate dielectric strength for each gas is expressed relative to that of the dielectric strength of nitrogen.

TABLE 1
Approximate Dielectric Strength of Gases Relative to Nitrogen
(unless units of kV/mm are indicated)

| | Dielectric | | Dielectric |
|---|---|---|---|
| Material | Strength | Material | Strength |
| Nitrogen, N₂ | 1.00 | Difluoromethane, CH₂F₂ | 0.79 |
| Hydrogen, H₂ | 0.50 | Trifluoromethane, CHF₃ | 0.71 |
| Helium, He | 0.15 | Bromochlorodifluoromethane, CF₂ClBr | 3.84 |
| Oxygen, O₂ | 0.92 | Chlorodifluoromethane, CHClF₂ | 1.11-1.40 |
| Air | 0.97 | Dichlorofluoromethane, CHCl₂F | 1.33-2.61 |
| Air (flat electrodes), kV/mm | 3.0 | Chlorofluoromethane, CH₂ClF | 1.03 |
| Air, kV/mm | 0.4-1.4 | Hexafluoroethane, C₂F₆ | 1.82-2.55 |
| Neon, Ne | 0.16-0.25 | Ethyne (Acetylene), C₂H₂ | 1.10-1.11 |
| Argon, Ar | 0.18 | Chloropentafluoroethane, C₂ClF₅ | 2.3-3.0 |
| Chlorine, Cl₂ | 1.55 | Dichlorotetrafluoroethane, C₂Cl₂F₄ | 2.52 |
| Carbon monoxide, CO | 1.02-1.05 | Chlorotrifluoroethylene, C₂ClF₃ | 1.82 |
| Carbon dioxide, CO₂ | 0.82-0.88 | 1,1,1-Trichloro-2,2,2-trifluoroethane | 6.55 |
| Nitrous oxide, N₂O | 1.24 | 1,1,2-Trichloro-1,2,2-trifluoroethane | 6.05 |
| Sulfur dioxide, SO₂ | 2.63-2.68 | Chloroethane, C₂H₅Cl | 1.00 |
| Sulfur monochloride, S₂ Cl₂ (at 12.5 Torr) | 1.02 | 1,1-Dichloroethane | 2.66 |
| Thionyl fluoride, SOF₂ | 2.50 | Trifluoroacetonitrile, CF₃CN | 3.5 |
| Sulfur hexafluoride, SF₆ | 2.50-2.63 | Acetonitrile, CH₃CN | 2.11 |
| Sulfur hexafluoride, SF₆, kV/mm | 8.50-9.8 | Dimethylamine, (CH₃)₂NH | 1.04 |
| Perchloryl fluoride, CIO₃F | 2.73 | Ethylamine, C₂H₅NH₂ | 1.01 |
| Tetrachloromethane, CCl₄ | 6.21-6.33 | Ethylene oxide (oxirane), | 1.01 |
| | | CH₃CHO | |
| Tetrafluoromethane, CF₄ | 1.01 | Perfluoropropene, C₃F₆ | 2.55 |
| Methane, CH₄ | 1.00-1.13 | Octafluoropropane, C₃F₈ | 2.19-2.47 |
| Bromotrifluoromethane, CF₃Br | 1.35-1.97 | 3,3,3-Trifluoro-1-propene, CH₂CHCF₃ | 2.11 |
| Bromomethane, CH₃Br | 0.71 | Pentafluoroisocyanoethane, C₂F₅NC | 4.5 |
| Chloromethane, CH₃Cl | 1.29 | 1,1,1,4,4,4-Hexafluoro-2-butyne, CF₃CCCF₃ | 5.84 |
| Iodomethane, CH₃I | 3.02 | Octafluorocyclobutane, C₄F₈ | 3.34 |
| Iodomethane, CH₃I at 370 Torr | 2.20 | 1,1,1,2,3,4,4,4-Octafluoro-2-butene | 2.8 |
| Dichloromethane, CH₂Cl₂ | 1.92 | Decafluorobutane, C₄F₁₀ | 3.08 |
| Dichlorodifluoromethane, CCl₂F₂ | 2.42-2.63 | Perfluorobutanenitrile, C₃F₇CN | 5.5 |
| Chlorotrifluoromethane, CClF₃ | 1.43-1.53 | Perfluoro-2-methyl-1,3-butadiene, C₅F₈ | 5.5 |
| Trichlorofluoromethane, CCl₃F | 3.50-4.53 | Hexafluorobenzene, C₆F₆ | 2.11 |
| Trichloromethane, CHCl₃ | 4.2-4.39 | Perfluorocyclohexane, C₆F₁₂, | 6.18 |
| | | (saturated vapor) | |
| Methylamine, CH₃NH₂ | 0.81 | | |

Other embodiments of the present subject matter are also contemplated. For example, in one embodiment, the gaseous environment is introduced following purging of contaminants from the interior of the housing. Purging, in various embodiments, includes injecting nitrogen or other gas mixture into the housing after forming a welded joint and prior to sealing the backfill port. The purging gas can include a mixture of gases such as helium, nitrogen, oxygen and argon.

The electronic circuit in the housing, in various embodiments, includes an defibrillator, a pacemaker, a pulse generator, a cardioverter as well as other therapeutic and monitoring medical devices.

### Conclusion

The above description is intended to be illustrative, and not restrictive. Many other embodiments will be apparent to those of skill in the art upon reviewing the above description.

1. An implantable device comprising:
a sealed housing;
a plurality of electrical components disposed within the housing and including at least one pair of adjacent electrically conductive paths wherein the paths are separated by a distance less than approximately 0.01 inches; and
a gas mixture of at least 1 percent sulfur hexafluoride disposed within the housing.

2. The device of claim 1 wherein the gas mixture includes at least 70 percent sulfur hexafluoride.

3. The device of claim 1 wherein the plurality of electrical components includes a defibrillator.

4. The device of claim 1 wherein the paths are separated by a distance less than approximately 0.004 inches.

5. The device of claim 1 wherein the gas mixture includes at least one of any combination of helium, argon, oxygen and nitrogen.

6. The device of claim 1 wherein the gas mixture is maintained at a pressure greater than one atmosphere.

7. The device of claim 1 wherein the gas mixture is maintained at a pressure greater than 0.1 pounds per square inch gage.

8. The device of claim 1 wherein the gas mixture is maintained at a pressure less than approximately 5.0 pounds per square inch gage.

9. The device of claim 1 wherein the sealed housing includes a welded joint.

10. The device of claim 1 wherein a breakdown voltage between the adjacent electrically conductive paths is greater than approximately 1000 volts.

11. The device of claim 1 wherein a breakdown voltage between the adjacent electrically conductive paths is greater than approximately 1800 volts.

12. A method comprising:
assembling a plurality of electrical components in an implantable housing wherein the plurality includes a pair of conductive paths separated by a distance of less than approximately 0.01 inches;
introducing a gaseous mixture of at least 1 percent sulfur hexafluoride to an interior of the housing; and
sealing the housing to prevent release of the gas mixture.

13. The method of claim 12 wherein introducing the gaseous mixture includes enveloping the plurality of electrical components in an atmosphere of greater than approximately 70 percent sulfur hexafluoride.

14. The method of claim 12 wherein sealing the housing includes welding the housing in an argon atmosphere.

15. The method of claim 12 wherein introducing the gaseous mixture includes:
drawing a first vacuum in the housing;
injecting a first atmosphere into the housing;
drawing a second vacuum in the housing; and
injecting a second atmosphere into the housing wherein the second atmosphere includes a mixture of helium.

16. The method of claim 15 wherein the first atmosphere differs from the second atmosphere.

17. The method of claim 15 wherein at least one of any combination of injecting the first atmosphere and injecting the second atmosphere includes injecting an atmosphere having greater than approximately 85 percent nitrogen.

18. The method of claim 15 wherein at least one of any combination of injecting the first atmosphere and injecting the second atmosphere includes injecting an atmosphere having greater than approximately 85 percent sulfur hexafluoride.

19. The method of claim 12 further including purging the interior of a contaminant gas.

20. The method of claim 19 wherein purging includes flushing with at least one of any combination of nitrogen and sulfur hexafluoride.

21. The method of claim 12 wherein introducing the gaseous mixture includes introducing at least one of any combination of helium, nitrogen, oxygen and argon.

22. The method of claim 12 wherein assembling the plurality of electrical components includes assembling a defibrillator.

23. The method of claim 12 wherein introducing the gaseous mixture includes pressurizing the housing to a pressure greater than one atmosphere.

24. The method of claim 23 wherein introducing the gaseous mixture includes pressurizing the housing to a pressure greater than approximately 0.1 pounds per square inch gage.

25. The method of claim 23 wherein introducing the gaseous mixture includes pressurizing the housing to a pressure less than approximately 5.0 pounds per square inch gage.

26. An implantable device comprising:
a sealed housing;
a plurality of electrical components disposed within the housing and including at least one pair of adjacent electrically conductive paths wherein the paths are separated by a distance less than approximately 0.01 inches; and
a gas mixture having a dielectric constant greater than unity disposed within the housing.

27. The device of claim 26 wherein the gas mixture includes greater than 1 percent sulfur hexafluoride.

28. The device of claim 26 wherein the gas mixture is maintained at a pressure greater than 0.1 pounds per square inch gage.

29. The device of claim 26 wherein the gas mixture is maintained at a pressure greater than 5.0 pounds per square inch gage.
